(19) United States
(12) Patent Application Publication
(10) Pub. No.: US 2002/0173752 A1
(43) Pub. Date: Nov. 21, 2002

(54) Method for reconstituting an injection liquid and an injection appliance for carrying out such a method

(76) Inventor: U1f Polzin, Leinfelden (DE)

Correspondence Address: WARE FRESSOLA VAN DER SLUYS &, ADOLPHSON, LLP, BRADFORD GREEN BUILDING 5, 755 MAIN STREET, P O BOX 224, MONROE, CT, 06468, US

(21) Appl. No.: 10/130,734

(22) PCT Filed: Nov. 29, 2000
(86) PCT No.: PCT/EP00/11916

(30) Foreign Application Priority Data
Feb. 16, 2000 (DE) 100 06 935.5

(51) Int. Cl. A61M 5/315; A61M 39/00

(52) U.S. Cl. 604/233; 604/237

(57) ABSTRACT

An injection device has a housing (42) for receiving a container (50) having a liquid (49; 226′). In said container (50), a piston (90) is displaceably arranged so that by means of pressure upon the distal end of said piston (90), liquid can be expelled from the container (50). Arranged displaceably in the housing (42) is a tappet (92), which is configured for contact against the distal end of the piston (90) and which enables transfer of a force to the piston (90) in the proximal direction. Associated with the tappet (92) and displaceable relative thereto is a member (91) which at its proximal end region can be joined to the piston (90) in such a way that it enables the transfer of a force in the proximal direction and the transfer of a force in the distal direction to the piston (90).

The invention concerns a method for reconstituting an injection liquid and an injection device that is suitable for reconstituting an injection liquid prior to injection.

Proteins in particular do not have a long lifetime in dissolved form, and must therefore be stored separately from the solvent and usually in lyophilized form. The active ingredient is not dissolved in the solvent until immediately before the first injection.

It is therefore an object of the invention to make available a new method for reconstituting an injection liquid, and an injection device for carrying out such a method.

According to the invention, this object is achieved by the subject matter of claim 1. A device of this kind is easy and uncomplicated to operate, since the patient understands and sees what is happening during reconstitution; and after reconstitution, the device is ready for an injection with no need for the patient to perform additional complex steps.

A method according to the present invention is the subject matter of claim 26. This method is simple and can be carried out even by laypersons, usually after appropriate instruction in a hospital or from a medical specialist.

Further details and advantageous developments of the invention are evident from the exemplary embodiment described below and depicted in the drawings, which is in no way to be understood as a limitation of the invention, and from the dependent claims.

In the Drawings:

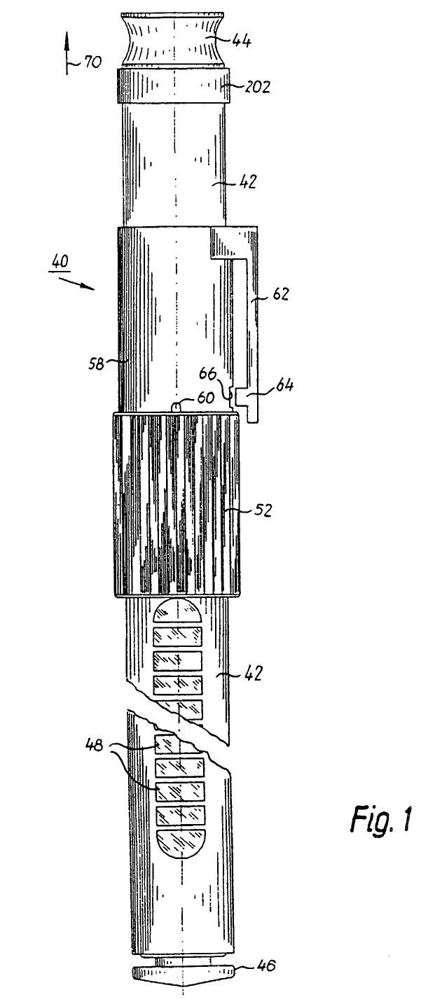

FIG. 1 is an external view of an injection device according to the present invention in the state in which it leaves the factory, i.e. prior to reconstitution of the lyophilisate;

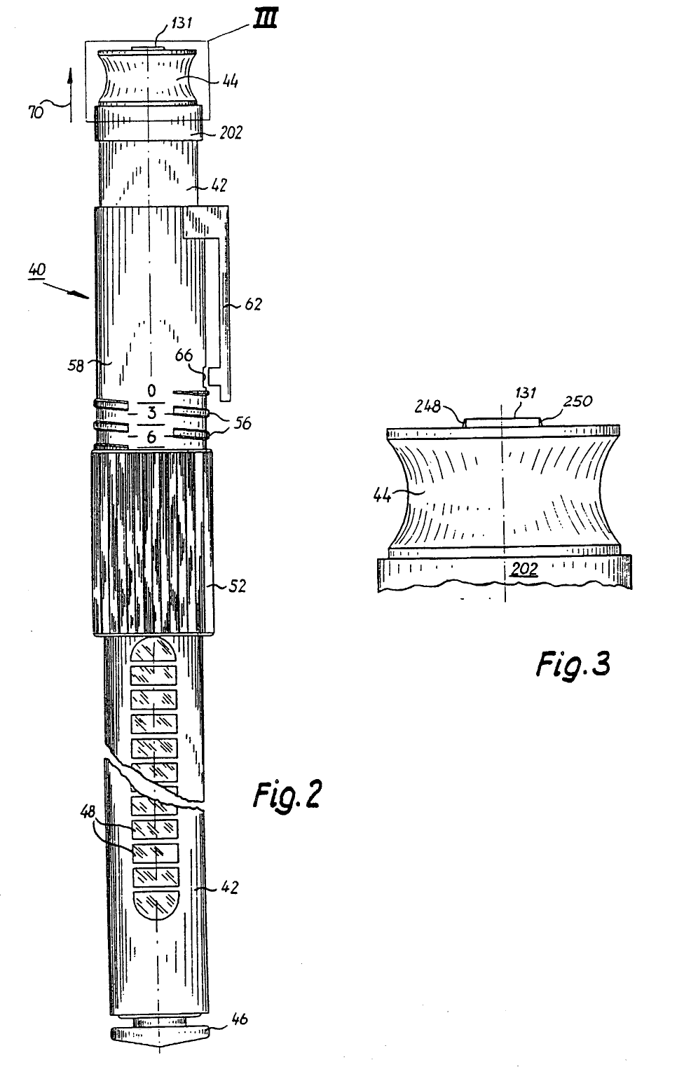

FIG. 2 is an external view of the injection device in the state after preparation for injections, i.e. after reconstitution of the lyophilisate, and after setting an injection dose of six units;

FIG. 3 is an enlarged depiction of portion III of FIG. 2;

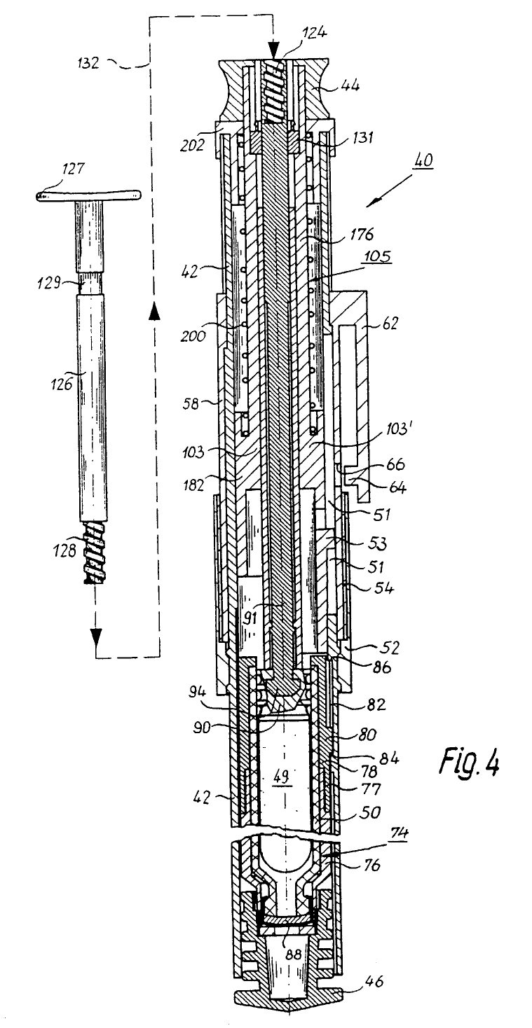

FIG. 4 is a longitudinal section through the injection device of FIG. 1, together with an extension rod 126 that is used during reconstitution;

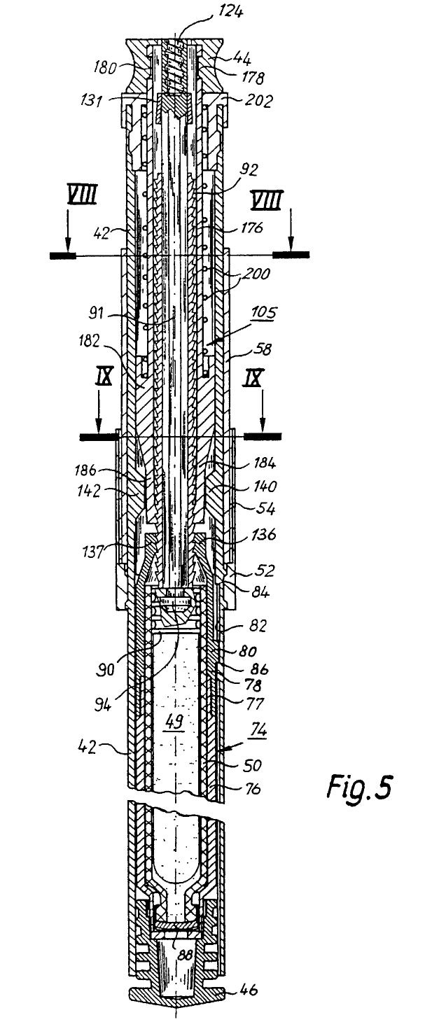

FIG. 5 is a longitudinal section similar to FIG. 4 but perpendicular to the plane of FIG. 4;

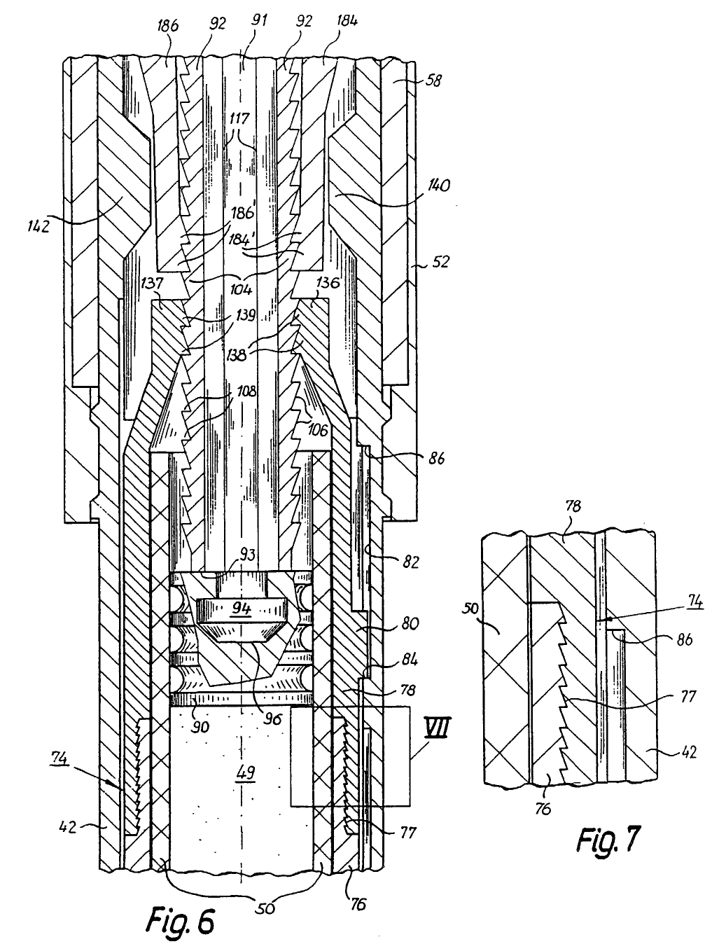

FIG. 6 is an enlarged depiction of the center part of FIG. 5;

FIG. 7 is an enlargement of the detail labeled VII in FIG. 6;

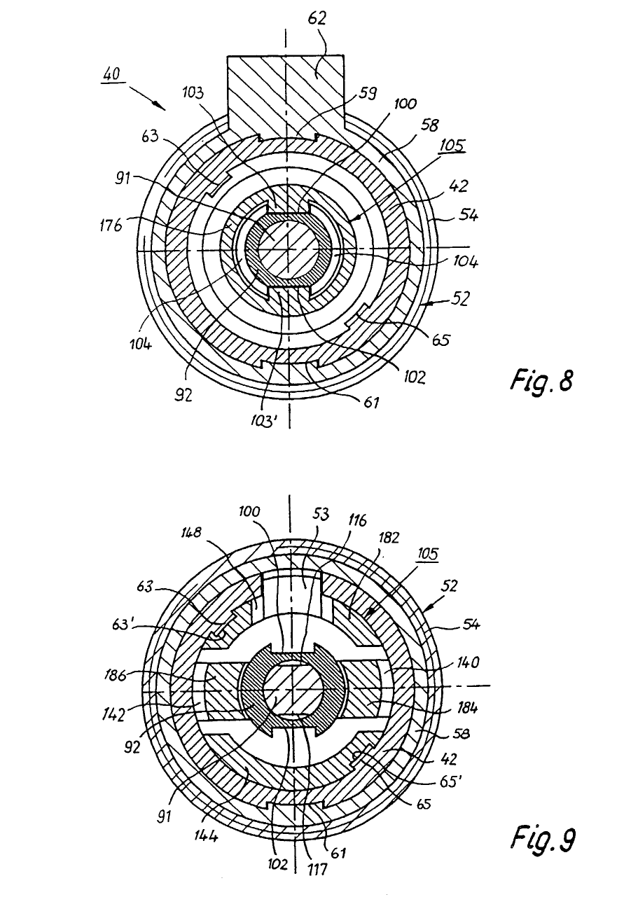

FIG. 8 is a section viewed along line VIII-VIII of FIG. 5;

FIG. 9 is a section viewed along line IX-IX of FIG. 5;

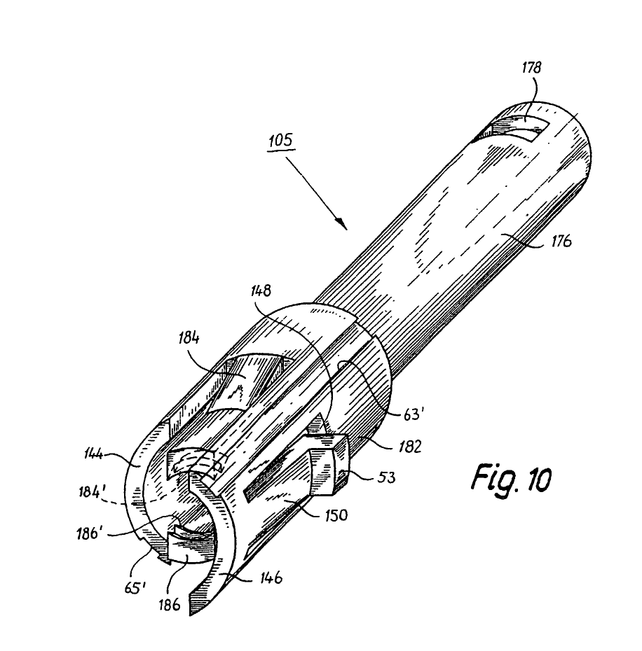

FIG. 10 is a three-dimensional depiction of an adjusting member used in the exemplary embodiment, which is displaced in the housing of the injection device in order to prepare an injection;

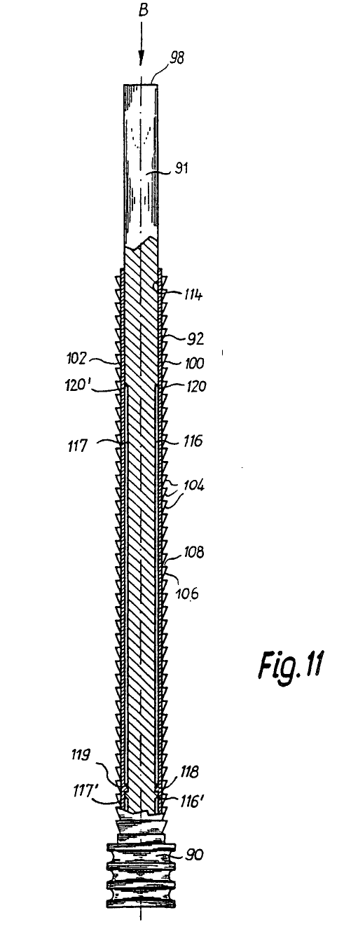

FIG. 11 is a partial longitudinal section through a toothed rod, viewed along line XI-XI of FIG. 13;

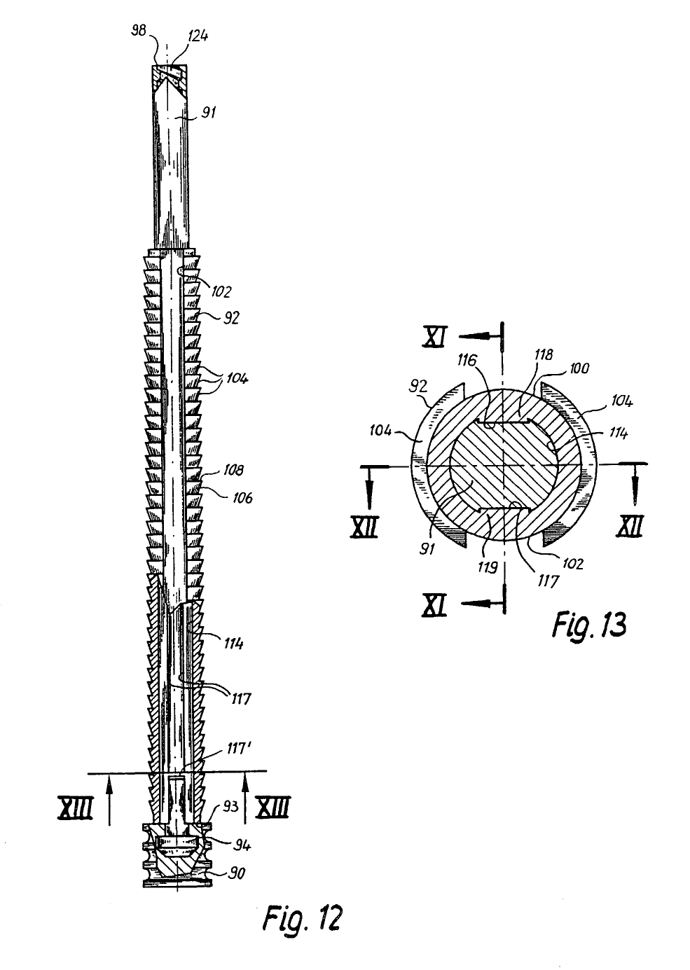

FIG. 12 is a partial longitudinal section similar to FIG. 11 but viewed along line XII-XII of FIG. 13; the upper part is shown unsectioned;

FIG. 13 is a section viewed along line XIII-XIII of FIG. 12;

FIG. 14 is a longitudinal section through a preferred joining part according to the invention;

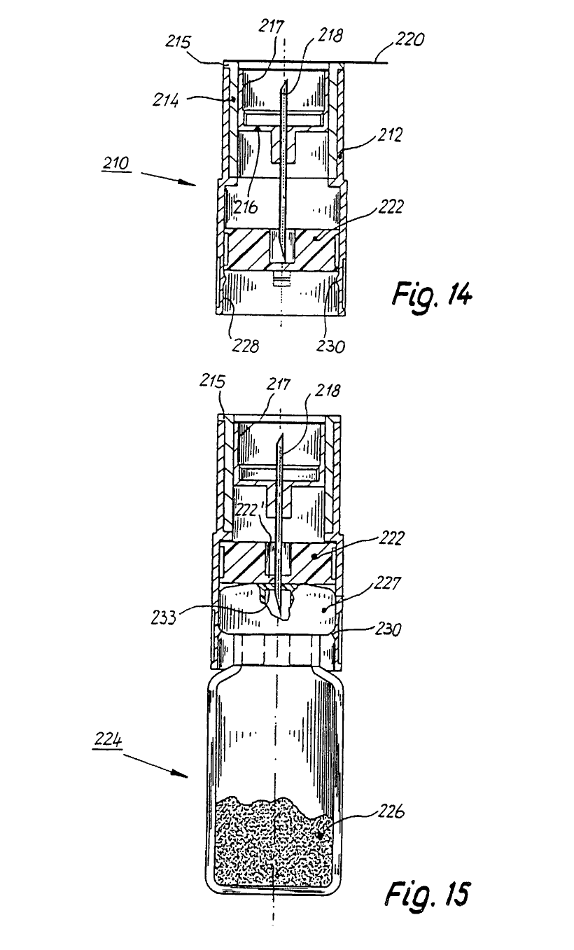

FIG. 15 is a section through the joining part of FIG. 14, to which a container having a lyophilized medication is joined;

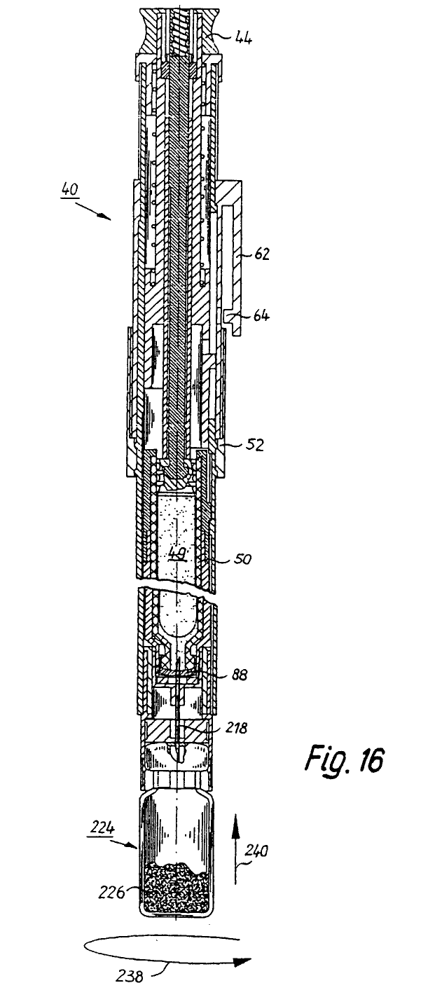

FIG. 16 is a longitudinal section similar to FIG. 4 through an injection device according to the present invention, onto which an arrangement as shown in FIG. 15 has been mounted;

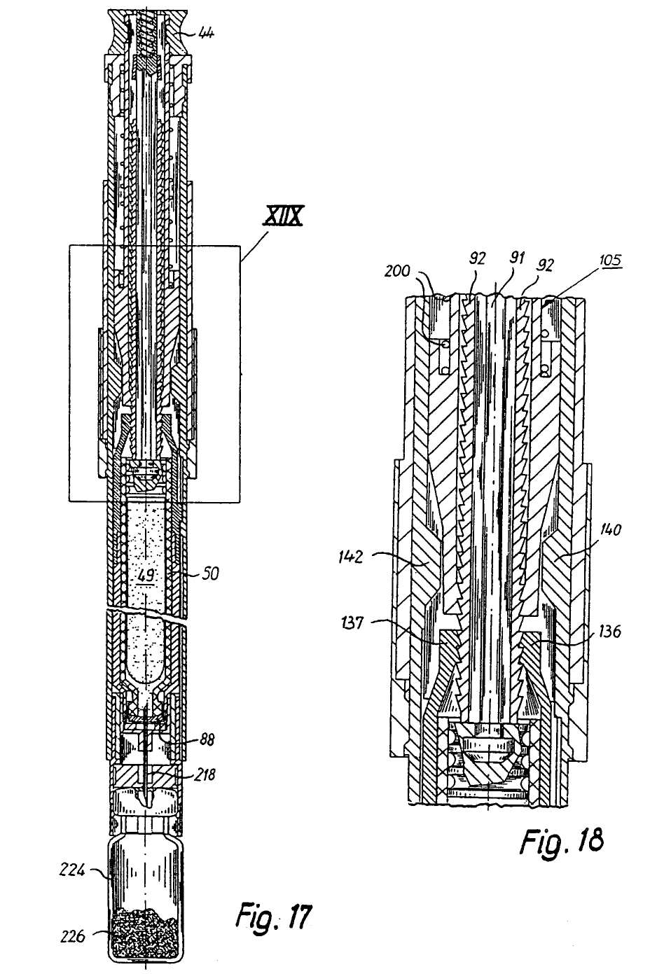

FIG. 17 is a longitudinal section through the arrangement of FIG. 16, but orthogonally to the section plane of FIG. 16;

FIG. 18 is an enlarged depiction of portion XIIX of FIG. 17;

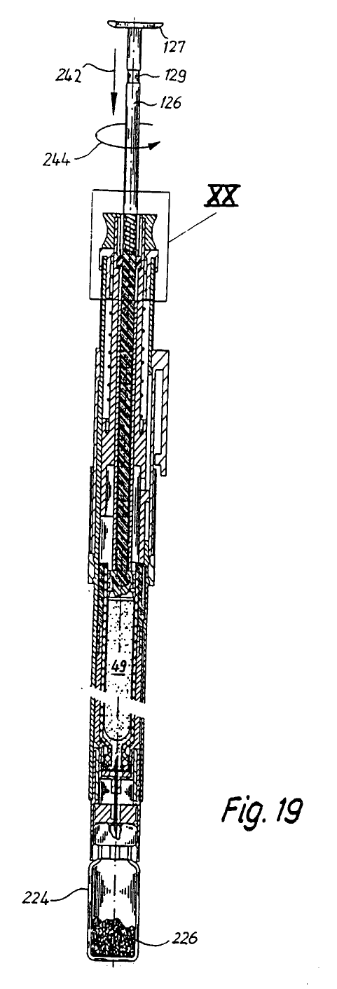

FIG. 19 is a depiction similar to FIG. 16, but after the mounting of extension rod 126 depicted in FIG. 4;

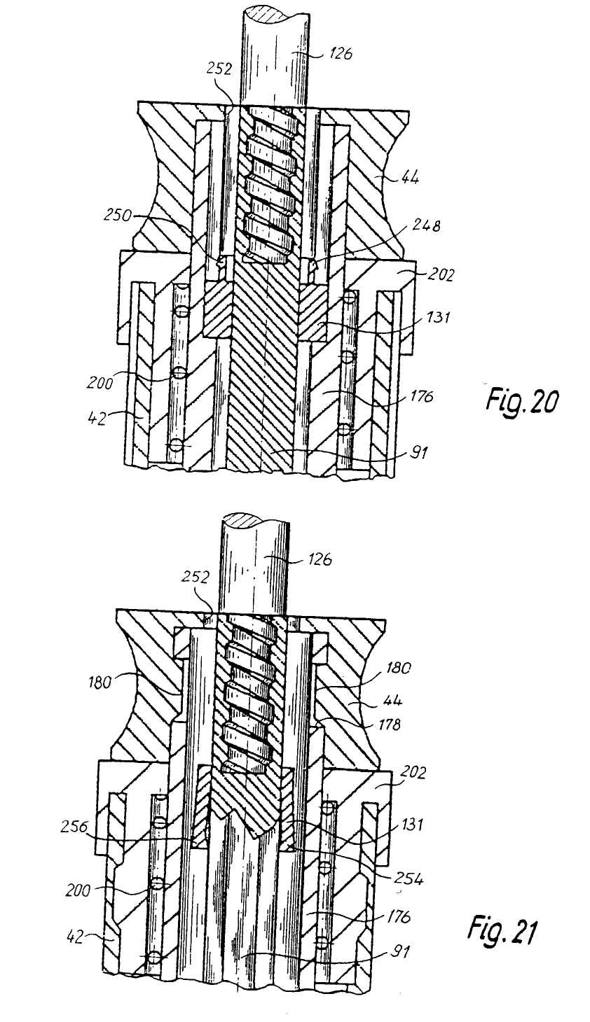

FIG. 20 is an enlarged depiction of the portion labeled XX in FIG. 19;

FIG. 21 is an enlarged depiction similar to FIG. 20 but rotated 90 degrees with respect to the section plane of FIG. 20;

FIG. 22 is a depiction similar to FIG. 19 but in the state in which extension rod 126 has been pushed in the direction of an arrow 242 completely into injection device 40 in order to expel much of liquid 49 out of the injection device into container 224 mounted thereon;

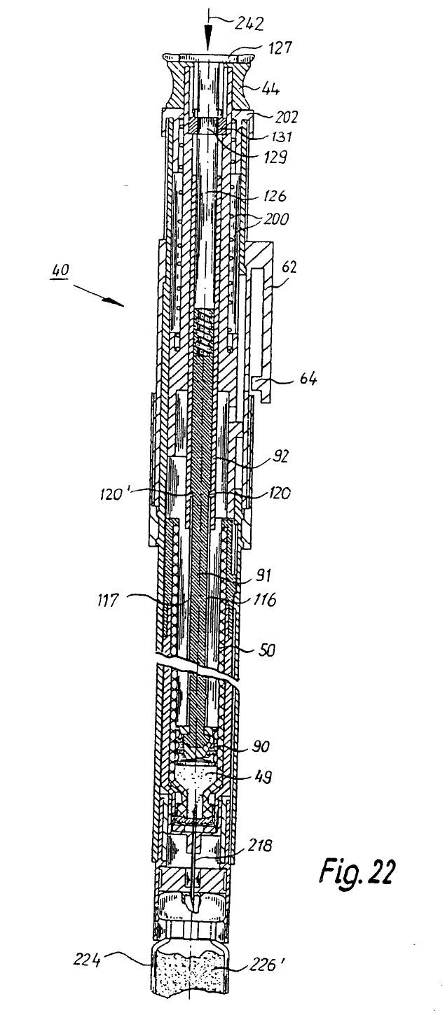

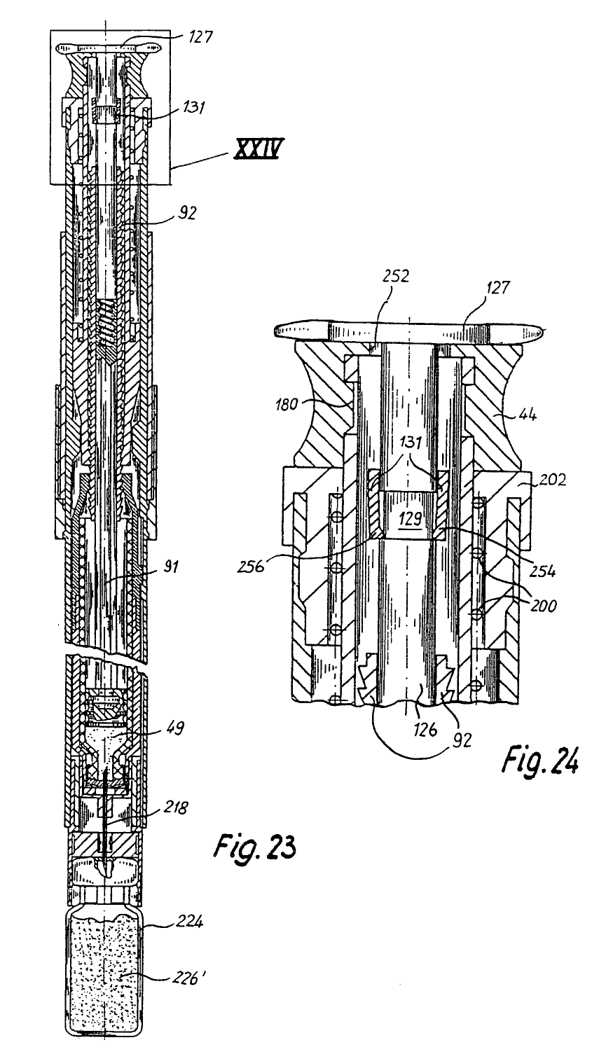

FIG. 23 is a depiction similar to FIG. 22 but in a section plane rotated 90 degrees with respect to FIG. 22;

FIG. 24 is an enlarged depiction of the portion labeled XXIV in FIG. 23, to explain the manner of operation of a signal member 131;

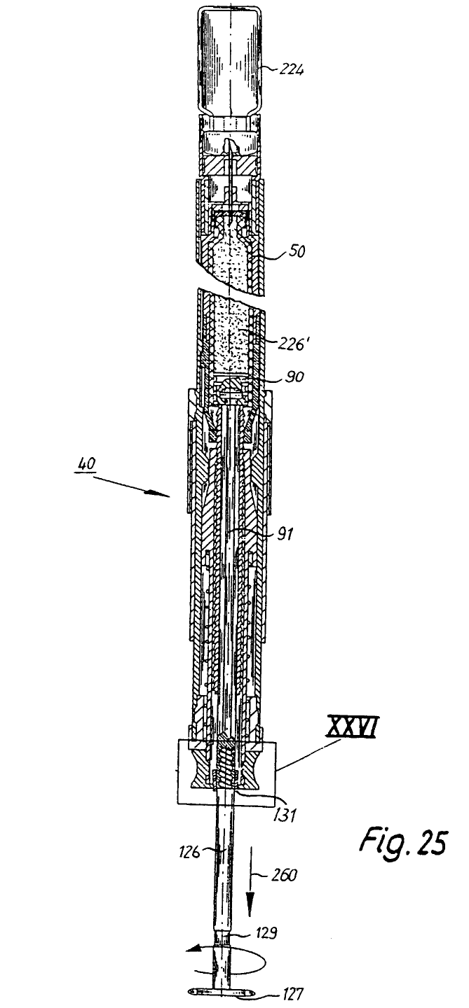

FIG. 25 is a depiction similar to FIG. 23, but after the reconstituted injection liquid 226′ has been drawn back into cartridge 50 of injection device 40 by pulling rod 126 in the direction of an arrow 260;

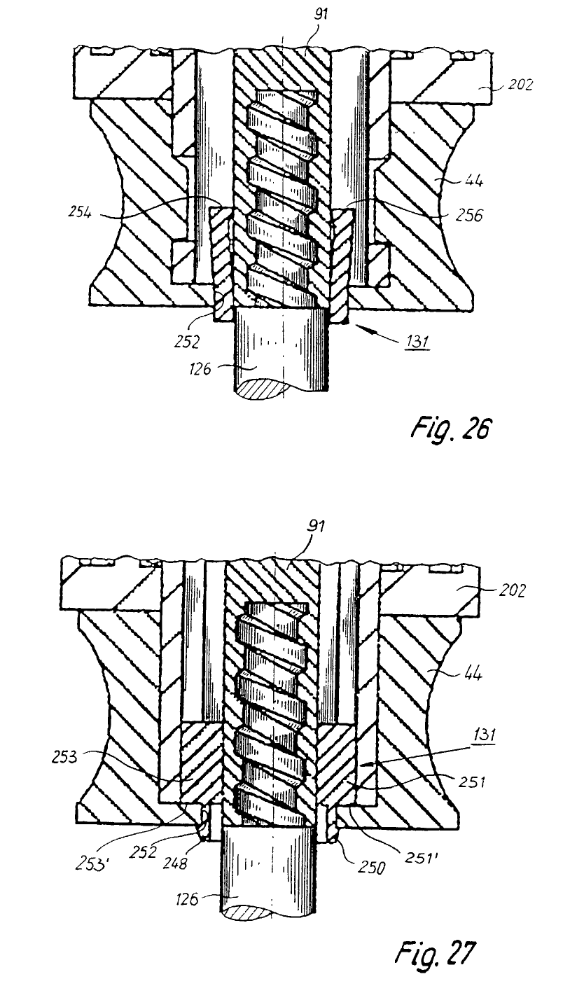

FIG. 26 is an enlarged depiction of the portion labeled XXVI in FIG. 25;

FIG. 27 is a depiction similar to FIG. 26, but in a section plane rotated 90 degrees with respect to FIG. 26;

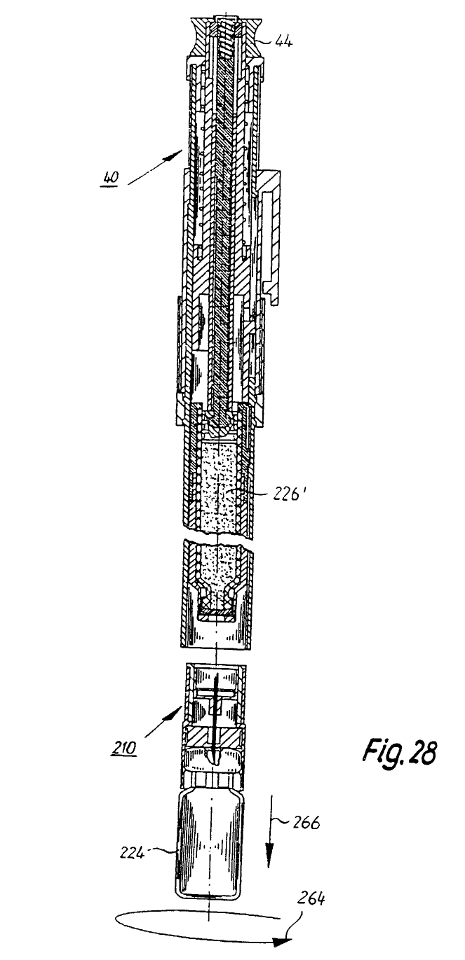

FIG. 28 is a section similar to FIG. 16, but after reconstitution and after removal of the empty container 224 and its adapter 210;

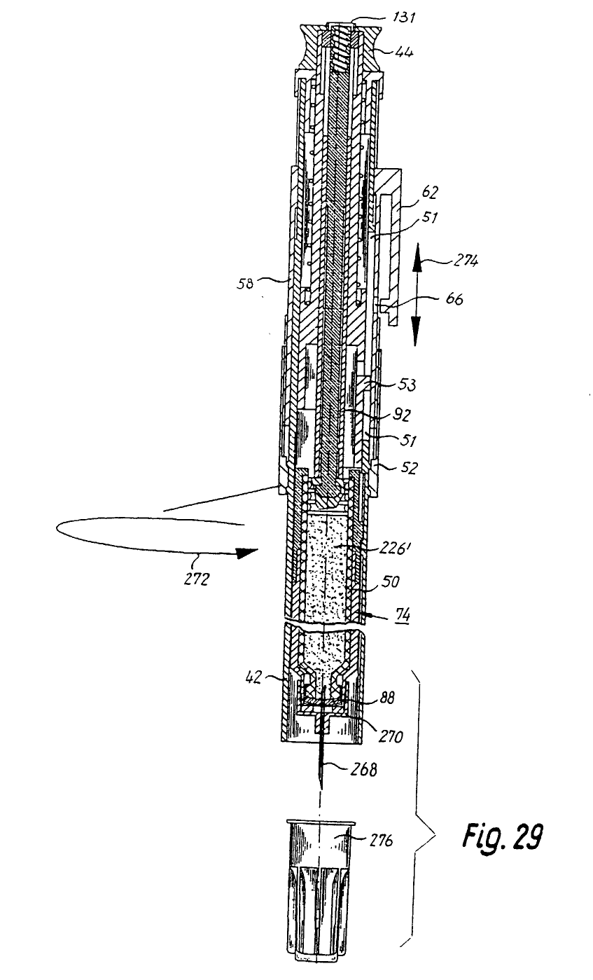

FIG. 29 is a depiction similar to FIG. 28, but after an injection needle 268 has been mounted and after the desired injection dose has been set;

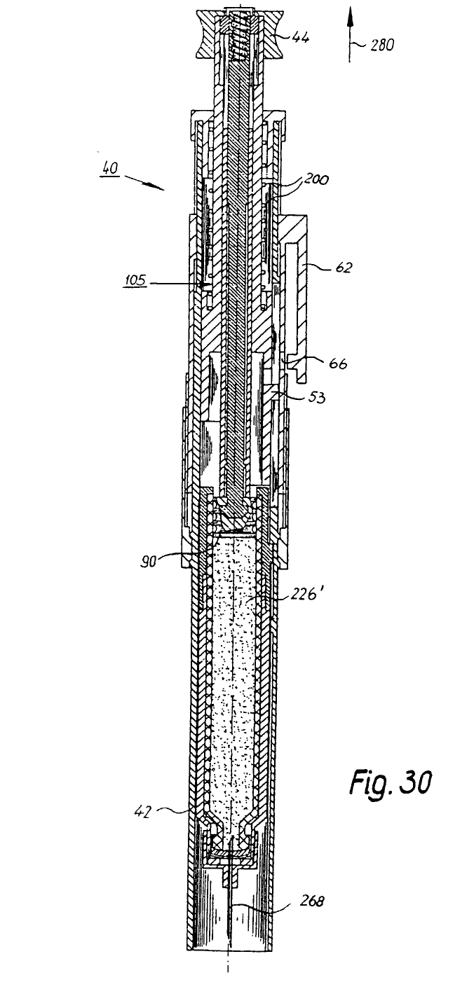

FIG. 30 is a depiction similar to FIG. 29 which shows the first part of the cocking operation with which the injection device is prepared for a subsequent injection;

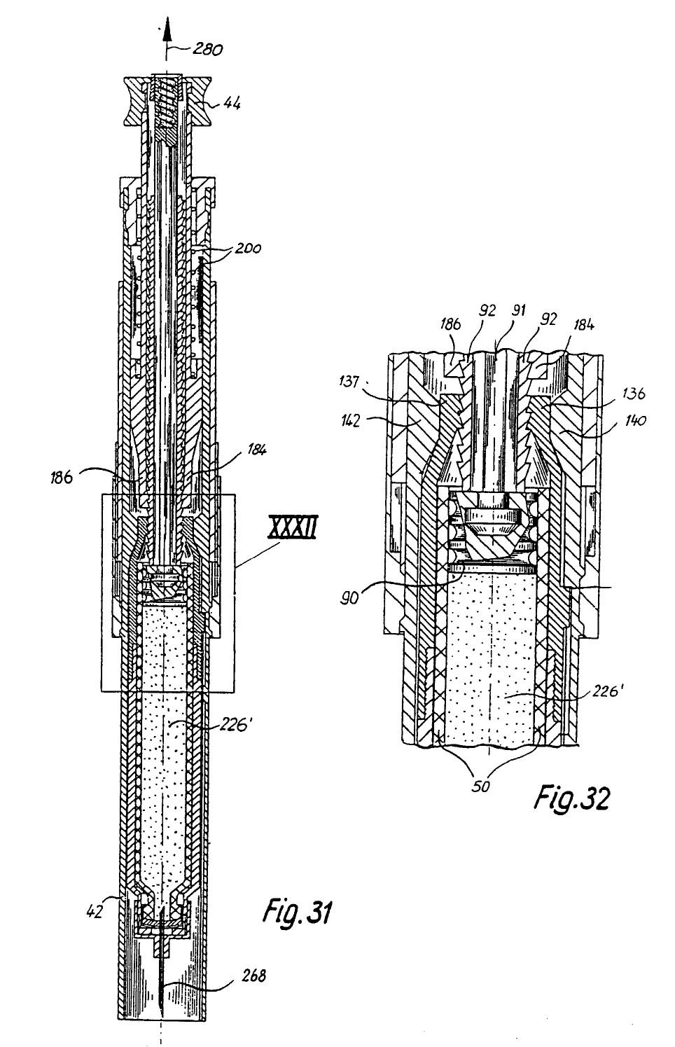

FIG. 31 is a sectioned depiction similar to FIG. 30, but in a section plane rotated 90 degrees relative to FIG. 30;

FIG. 32 is an enlarged depiction of the portion labeled XXXII in FIG. 31;

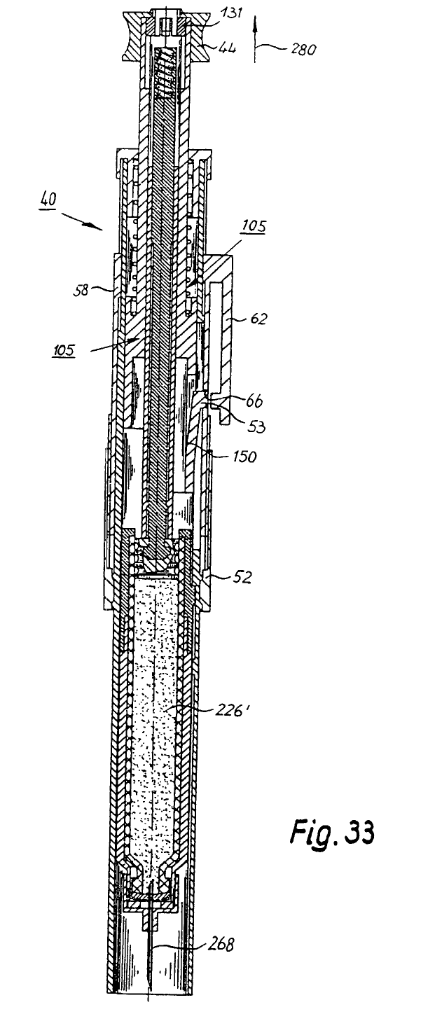

FIG. 33 is a depiction similar to FIG. 30 which shows the state of the injection device after completion of the cocking operation; the device is now ready for an injection;

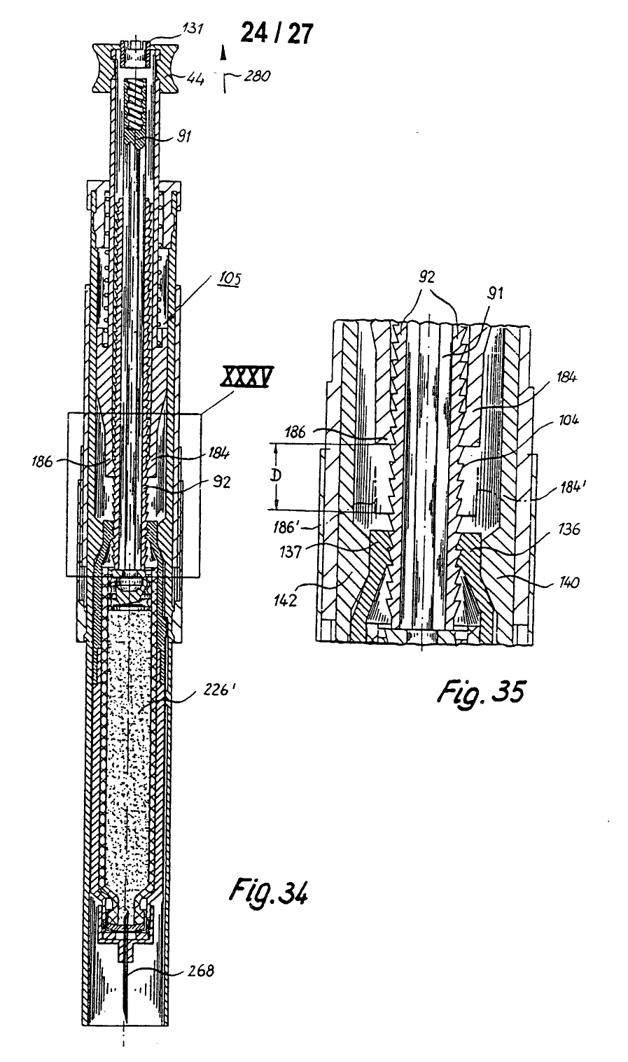

FIG. 34 is a sectioned depiction similar to FIG. 33 but in a section plane orthogonal to FIG. 33;

FIG. 35 is an enlarged depiction of the portion labeled XXXV in FIG. 34;

FIG. 36 is a longitudinal section similar to FIG. 29, but after an injection has been performed (note the change in the position of toothed rod 92 as compared to FIG. 29);

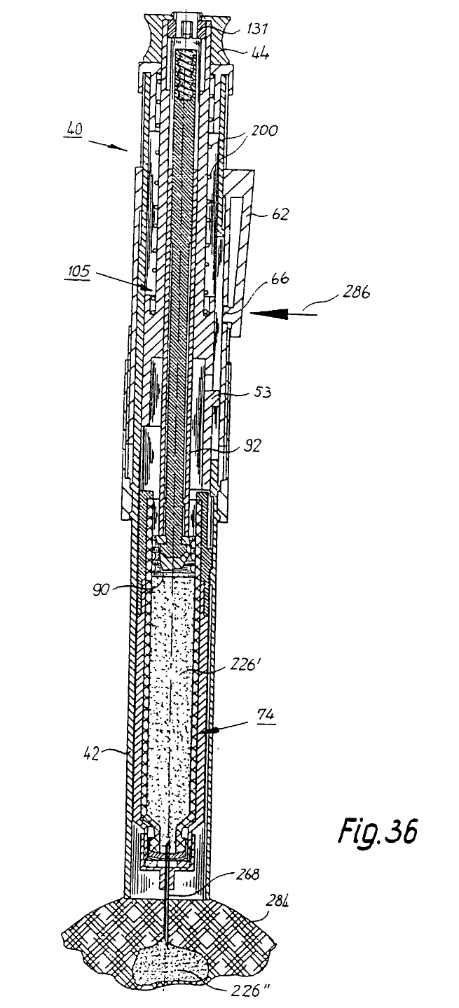

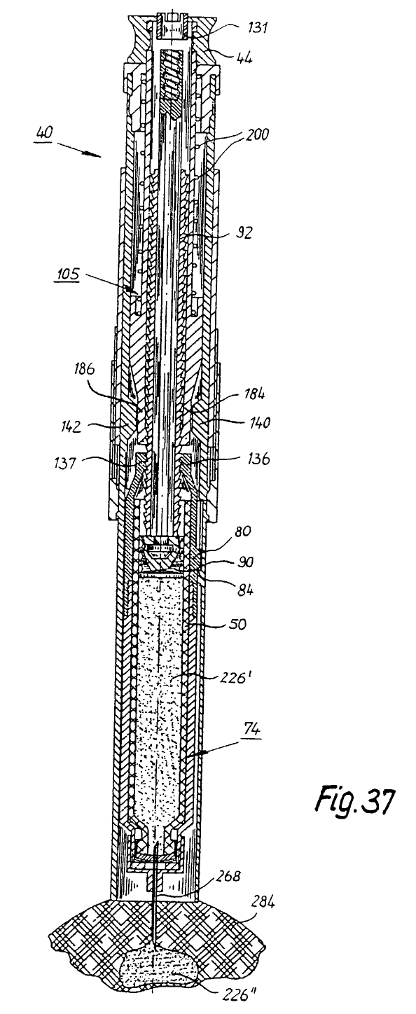

FIG. 37 is a sectioned depiction similar to FIG. 26, but in a section plane rotated 90 degrees as compared to FIG. 36;

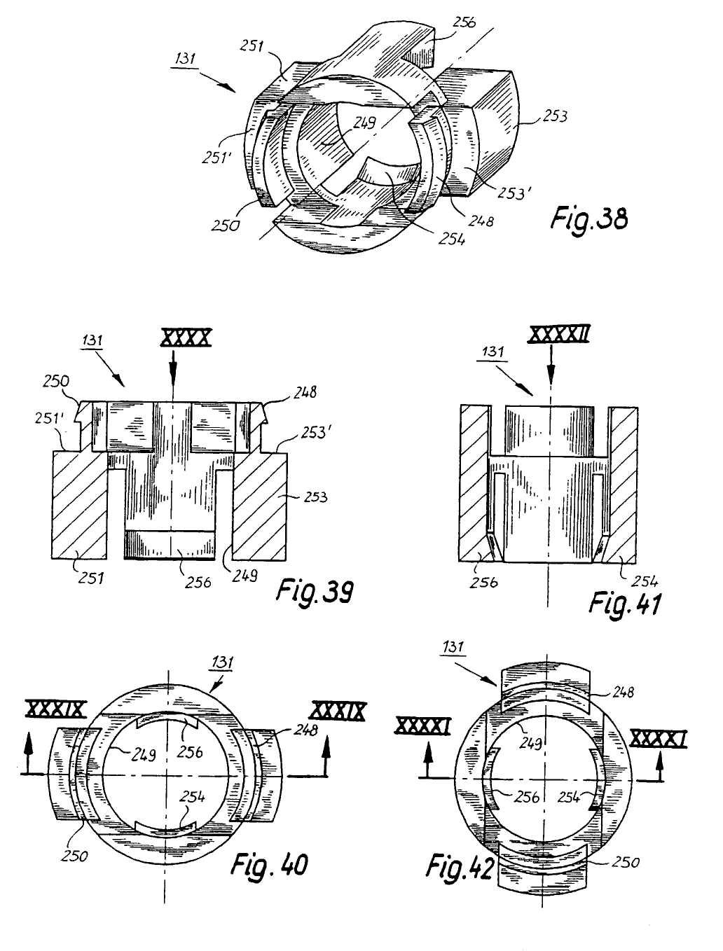

FIG. 38 is a three-dimensional depiction of a signal part used in the exemplary embodiment to indicate reconstitution;

FIG. 39 is a section through the signal part, viewed along line XXXIX-XXXIX of FIG. 40;

FIG. 40 is a plan view of the signal part, viewed in the direction of arrow XXXX of FIG. 39;

FIG. 41 is a section through the signal part, viewed along line XXXXI-XXXXI of FIG. 42; and

FIG. 42 is a plan view, viewed in the direction of arrow XXXXII of FIG. 41.

FIG. 1 shows, at enlarged scale, an injection device 40 in the state in which it leaves the manufacturer's factory. It resembles an oversized fountain pen, and has an elongated tubular housing 42. Located at the distal end (i.e. at the top in FIG. 1) is an actuation knob 44 that serves to cock the device prior to an injection (cf. FIGS. 30 through 35); and at the bottom, i.e. at the proximal end, where a covering cap 46 is depicted, a needle is mounted before use (cf. FIG. 29).

As is usual in medicine and anatomy, the terms “distal,” “medial,” and “proximal” are used as follows:

Proximal: the end toward the patient, i.e. the end having the needle (at the bottom in FIG. 1).

Distal: the end away from the patient, i.e. in FIG. 1 the end having actuation knob 44.

Medial: directed toward the center axis of device 40; near the center axis.

Located in the proximal part of housing 42 is a window 48 through which the patient can see how much liquid 49 is present in a cartridge 50 therein (FIG. 4). Located in the distal part of housing 42, is an elongated opening 51 (FIG. 4) which serves for axial guidance of a resilient latching member 53.

A dose-setting wheel 52 is arranged, rotatably but axially nondisplaceably, on the center part of housing 42. It has an internal thread 54 (FIG. 4) that engages into an external thread (coarse thread) 56 (FIG. 2) of a dose-setting part 58 which is guided axially displaceably but nonrotatably in longitudinal grooves 59, 61 of housing 42 (cf. FIG. 8). When dose-setting wheel 52 is rotated, dose-setting part 58 is displaced axially, as is evident from a comparison of FIGS. 1 and 2; i.e. FIG. 1 shows a dose setting of “0” (as evidenced by the “0” depicted at 60), and FIG. 2 shows a dose setting of “6”. The injection dose can thus be set by rotating dose-setting wheel 52. If the injection dose that has been set is not modified, the same dose is then injected at each injection. This is referred to as “steady dose injection.” The dosage can, however, be modified as desired prior to each injection provided dose-setting wheel 52 has not been immobilized.

Provided on dose-setting part 58 is a resilient clip 62 that serves to trigger an injection. At its free end it has a medial protrusion 64, projecting toward device 40, that lies opposite a latch opening 66 of dose-setting part 58. A comparison of FIGS. 1 and 2 shows how this opening 66, together with clip 62, is displaced in the distal direction as the dose is increased. Latch opening 66 aligns with elongated opening 51 of housing 42.

FIG. 1 shows at the distal end (i.e. at the top) an arrow 70 to indicate that actuation knob 44 can be displaced in the direction of said arrow 70 in the distal direction. Knob 44 is not rotatable, as will become evident from the description below.

FIG. 4 is a longitudinal section through injection device of FIG. 1, and FIG. 5 is a similar longitudinal section but in a plane perpendicular to FIG. 4.

A transparent cartridge holder 74, which is assembled from a proximal part 76 and a distal part 78 and retains cartridge 50 in its interior, is located in the proximal part of housing 42. Parts 76, 78 are joined to one another by a micro-ratchet system 77 that is shown greatly enlarged in FIGS. 6 and 7. It permits simple yet highly accurate setting of the length of cartridge holder 74 for adaptation to cartridge 50 and its fill level. Said fill level can vary as a result of inaccuracies during manufacture and during filling of the cartridges. FIGS. 6 and 7 show the shortest possible length of cartridge holder 74.

Part 78 has radial protrusions 80 that are guided in corresponding longitudinal grooves 82 of housing 42 and are displaceable therein between two stops 84, 86. As a result, cartridge holder 74 is not rotatable relative to housing 42, and is axially displaceable only to a limited extent. FIG. 6 shows protrusion 80 at larger scale.

In the state as shown in FIGS. 1 and 4 through 6, liquid 49 in cartridge 50 is only a solvent, e.g. a sodium chloride solution. This solvent must still have added to it an active ingredient, which usually is present as a lyophilisate in a sterile container (cf. container 224 in FIG. 15 with lyophilisate 226). The manner in which this occurs will be described in detail below. This is called the “reconstitution” of liquid 49, in other words, its conversion into a medication that can subsequently be injected by the patient. This requires that the injection device be suitably “prepared” before it can be utilized by the patient.

The reason for separate storage of the solvent and active ingredient is that the reconstituted liquid has only a limited life, for example two to three weeks. Reconstitution is usually performed by specialized personnel (a nurse or physician), but can also be performed by the patient him- or herself if he or she has been appropriately instructed. A substantial advantage of an injection device according to the present invention is simple and easily understandable reconstitution, which can be carried out without difficulty even by a layperson.

In the usual way, cartridge 50 has at its proximal end a rubber membrane 88 that can be penetrated by a needle (cf. FIG. 29). At its distal end it has a rubber piston 90 on which is mounted the proximal end of an actuation member 91 that is partially depicted in FIG. 6 in a detail enlargement. For that purpose, the proximal end of actuation member 91 is snapped with a mushroom-shaped protrusion 94 into a recess 96, complementary thereto, of piston 90 (cf. FIG. 6). Alternatively, piston 90 can also be joined to actuation member 91 by way of a threaded connection.

In this exemplary embodiment, actuation member 91 is guided axially displaceably and nonrotatably in the interior of a toothed rod 92 (FIGS. 11 to 13), serving as tappet, which rests with its proximal end 93 against the distal end of piston 90 but is not immovably joined to it. Toothed rod 92 serves to carry out injections; it can displace piston 90 only in the proximal direction, but not in the distal direction.

Actuation member 91 serves to carry out a reconstitution, and it can therefore displace piston 90 in both the proximal and the distal direction. Upon distal displacement of piston 90, proximal end 93 of toothed rod 92 serves as a stop (cf. FIG. 11).

FIG. 11 shows a partial longitudinal section through actuation member 91 and toothed rod 92, viewed in the direction of arrow XI-XI of FIG. 13. Actuation member 91 has a cylindrical outer periphery in its distal region, and has on a portion of its longitudinal extension two oppositely located longitudinal grooves 116, 117 that are guided by protrusions 118, 119 on the inner side of a substantially cylindrical recess 114 of toothed rod 92. Longitudinal grooves 116, 117 extend in the distal direction as far as distal groove ends 120, 120′ (FIG. 22), and when the latter strike against stops 118, 119 during reconstitution, this limits the movement of actuation member 91 in the proximal direction (cf. FIG. 22). Conversely, protrusions 116′, 117′ are located in longitudinal grooves 116, 117 and limit the movement of actuation member 91 in the distal direction when they strike against stops 118, 119, as depicted in FIG. 11. This type of movement in the distal direction takes place at the end of a reconstitution.

As depicted in FIGS. 11 through 13, piston 90 is mounted on the mushroom-shaped proximal end 94 of actuation member 91 in such a way that it follows the movements of actuation member 91 in the proximal and distal directions.

As shown in FIG. 13, toothed rod 92 has a substantially round cross section with two oppositely located longitudinal grooves 100, 102 for axial longitudinal guidance. These grooves are guided in inwardly projecting protrusions 103, 103′ (FIGS. 4, 8) of an adjusting member 105 that in turn is guided nonrotatably by protrusions 63, 65 (FIGS. 8, 9) and longitudinal grooves 63′, 65′ (FIGS. 8, 9, 10) in the interior of housing 42, so that toothed rod 92 is displaceable relative to housing 42 not rotatably but only axially.

FIGS. 11 and 12 show teeth 104 of toothed rod 92 at enlarged scale. They have the sawtooth shape that is characteristic of a toothed rod, i.e. proximal part 106 of a tooth 104 has a flat slope, whereas distal part 108 has a steep slope.

The distal end of actuation member 91 has an internal thread 124 (FIGS. 4 and 12) into which extension rod 126 can be screwed with its external thread 128, as indicated in FIG. 4 by a dashed line 132, in order to extend actuation member 91 accordingly during reconstitution. Extension rod 126 has an actuation knob 127 on its distal end; external thread 128 is located at the proximal end. It also has in the region of its distal end an annular groove 129 which coacts with a signal member 131 (FIGS. 38 through 42) that is arranged axially displaceably on actuation member 91. Signal member 131 is activated in the context of a reconstitution, and after completion of the reconstitution signals that injection device 40 contains reconstituted injection liquid and is ready for an injection.

FIGS. 38 through 42 show details of signal member 131. Its construction is easily visible from the (greatly enlarged) three-dimensional depiction of FIG. 38. Its general structure is that of a ring whose internal recess 249 slides on the outer side of actuation member 91 or extension rod 126. From this ring, two snap-lock hooks 248, 250 extend in the distal direction and two resilient segments 254, 256 extend in the proximal direction. Signal member 131 additionally has two radially outwardly projecting segments 251 and 253, which after reconstitution come into contact with their distal end faces 251′ and 253′ respectively (in the position shown in FIG. 27) against rotary knob 44, while snap-lock hooks 248, 250 snap into rotary knob 44 as shown in FIGS. 2 and 3. This position of signal member 131 indicates that the device contains reconstituted injection liquid.

As shown in FIGS. 5 and 6, two resilient parts 136, 137 are located at the distal end region of cartridge holder 74; they can also be referred to as clamping jaws, and are equipped on their medial side with ratchet teeth 138 and 139, respectively, for engagement into teeth 104 of toothed rod 92. In the position shown in FIG. 5, resilient parts 136, 137 rest resiliently against toothed rod 92, i.e. have a preload in the medial direction, but permit a displacement of toothed rod 92 in the proximal direction because they can deflect outward oppositely to the medial direction.

The sectioned depiction of FIG. 8 shows how dose-setting part 58, which carries trigger clip 62, is guided in longitudinally displaceable fashion in two longitudinal grooves 59, 61 of housing 42.

Housing 42 has on its inner side two protrusions 63, 65 extending in the longitudinal direction. These serve to guide adjusting member 105′ (depicted three-dimensionally in FIG. 10) in the longitudinal direction and to prevent it from rotating. As depicted in FIG. 10, adjusting member 105 comprises one smaller-diameter tubular part 176 and one larger-diameter part 182. The latter has longitudinal grooves 63′, 65′ which are guided by protrusions 63, 65 of housing 42.

At its distal end region, tube 176 has two latch openings 178 that, as shown in FIG. 5, serve to secure radial protrusions 180 of actuation knob 44. Tube 176 has on its inner side two axially extending protrusions 103, 103′ (FIG. 8) that engage into longitudinal grooves 100, 102 of toothed rod 92 and prevent the latter from rotating.

Segment 182 of adjusting member 105, which is depicted in three-dimensional form in FIG. 10, has one shorter half-shell 144 and one longer half-shell 146 which extend away from tube 176 in the proximal direction. Located in half-shell 146 is a U-shaped recess 148 that defines a resilient tongue 150 at whose distal, free end is located the outwardly projecting latching member 53 which is caused, before an injection, to snap into latch opening 66 of dose-setting part 58 (cf. FIG. 33). In the position shown in FIGS. 5 and 9, it rests with a resilient preload against the inner side of dose-setting part 58 and as a result is displaced radially inward. In the position shown in FIG. 10, resilient tongue 150 is deflected outward. This corresponds to the position depicted in FIG. 33.

Part 182 also has two resilient clamping jaws 184, 186 that lie between half-shells 144, 146 and are equipped on their sides facing toward toothed rod 92 with ratchet teeth 184′, 186′. The latter are approximately complementary in configuration to teeth 104 of toothed rod 92, and engage with elastic preload into said teeth 104.

A distal shoulder of part 182 serves as abutment for the proximal end of an injection spring 200 (not depicted in FIG. 8) whose distal end rests against a closure cap 202 that, in the manner depicted in FIG. 21, is snap-locked into the distal end of housing 42 and forms a guide for tube 176.

An adapter 210 that is depicted in FIGS. 14 and 15 is used for reconstitution. It has an approximately cylindrical housing 212 in which a hollow needle holder 214 is mounted, for example by press-fitting, adhesive bonding, or welding. The latter has an upper flange 215 that extends radially over the upper rim of housing 212. A standard hollow needle support 216, with its hollow needle 218, is mounted in suitable fashion in hollow needle holder 214. Hollow needle holder 214 is sealed at the top in sterile fashion with a peelable film 220 that is applied detachably onto flange 215. An internal thread 217 is provided in hollow needle support 216 for mounting onto injection device 40.

Located in the lower part of housing 212 is a displaceable rubber piston 222 that, in the unused state, covers the lower end of hollow needle 218 in sterile fashion, while the upper end is covered by peelable film 220. This allows sterile storage of hollow needle 218.

As FIG. 15 shows, a container 224 that contains a lyophilisate 226 is pressed with its cap 227 into lower recess 228 of housing 212 and snaps into place there on a latch protrusion 230. It thereby displaces rubber piston 222 upward, and the latter, as depicted, is penetrated at a thin middle portion 222′ by the lower end of hollow needle 218, which then penetrates a rubber membrane 233 in cap 227. Peelable film 220 is then pulled off, and the arrangement as shown in FIG. 15 can, by means of internal thread 217, be screwed like an injection needle onto injection device 40 (cf. FIGS. 16 and 17). In the process, the upper (in FIGS. 14 and 15) end of hollow needle 218 penetrates through rubber membrane 88 of cartridge 50 and creates a connection from it to container 224, so that liquid 49 can flow out of cartridge 50 through hollow needle 218 into container 224.

FIG. 16 illustrates how the arrangement shown in FIG. 15 is screwed onto the proximal end of injection device 40, symbolized by a rotation arrow 238 and an arrow 240. In this context, needle 218 penetrates with its distal end through rubber membrane 88 of cartridge 50 and thereby connects the interiors of cartridge 50 and container 224.

FIG. 17 shows a longitudinal section that extends perpendicular to the plane of FIG. 16.

FIG. 18 shows an enlarged portion XVIII of FIG. 17. It is evident that device 40 is in the same position as in FIGS. 4 and 5.

FIG. 19 shows extension rod 126 being screwed on, by being attached in the direction of a proximal arrow 242 and rotated in the direction of a rotation arrow 244. The remainder is identical to FIG. 16.

FIG. 20 shows an enlarged depiction of portion XX of FIG. 19; FIG. 21 shows the same portion but in a section that extends perpendicular to the section plane of FIG. 20.

Signal member 131, which is arranged axially displaceably on actuation member 91, is apparent. As shown in FIGS. 20 and 39, it has at its distal end two barbs 248, 250 which are configured so that they can snap into an upper opening 252 of actuation knob 44, as shown e.g. in FIGS. 3 and 33. Signal member 131 moreover has two resilient segments 254, 256 (FIG. 21) whose proximal ends are configured so that they can snap releasably into the relatively flat annular groove 129 of extension rod 126, as shown in FIG. 24.

As shown in FIG. 22, extension rod 126 is then pushed in the direction of arrow 242 in the proximal direction into injection device 40, so that piston 90 is displaced correspondingly and much of liquid 49 is expelled through needle 218 into container 224, where said liquid 49 dissolves lyophilisate 226 to yield an injection solution 226′.

As FIGS. 23 and 24 clearly show, the position of toothed rod 92 does not change during this operation. Parts 254, 256 of signal member 131, which are pressed resiliently in the medial direction, snap with their proximal ends releasably into annular groove 129.

As shown in FIG. 25, injection device 40 is then turned upside down and extension rod 126 is pulled out of injection device 40 in the direction of an arrow 260 in the distal direction by pulling on actuation member 127. Actuation member 91 thus pulls along with it piston 90 that is joined to it, and sucks reconstituted liquid 226′ back into cartridge 50, where said liquid 226′ mixes with the remainder of liquid 49.

Since signal member 131 in this process (see FIGS. 26 and 27) strikes with its protrusions 251, 253 against actuation knob 44, it can follow the distal motion of extension rod 126 over only a portion of its travel, i.e. it is at first carried along by annular groove 129 and ultimately snaps into opening 252 of actuation knob 44. Parts 248, 250 have a suitable signal color, and indicate by their presence and visibility that injection device 40 contains reconstituted injection liquid 226′ and is ready for use.

As shown in FIG. 28, the empty container 224 is then, together with adapter 210, unscrewed (rotation arrow 264) and removed (arrow 266) from injection device 40.

Then, as shown in FIG. 29, an injection needle (hollow needle) 268 is screwed by means of its hollow needle support 270 onto the proximal end of cartridge holder 74, so that its distal end penetrates through rubber membrane 88 of cartridge 50. The injection dose is then set by rotating dose-setting wheel 52 (rotation arrow 272) as already explained with reference to FIGS. 1 and 2, thus causing dose-setting part 58 with its clip 62 and latch opening 66 to be correspondingly displaced; this is symbolized by arrow 274. A comparison with FIGS. 1 and 2 illustrates what happens during the setting operation.

Note that in the position shown in FIG. 29, i.e. before injection device 40 is cocked, needle 268 projects downward out of housing 42. The device also assumes this position after an injection. FIG. 29 also shows a needle safety cap 276 which is used for sterile installation of needle 268 and which, in the position shown in FIG. 29, can be left on needle 268 on order to protect it and the user.

FIGS. 30, 31, and 32 show the first part of the cocking operation with which injection device 40 is prepared for a subsequent injection. For this purpose, the user pulls on knob 44 in the distal direction in the direction of an arrow 280 (FIGS. 30, 31), causing needle 268 to be pulled into housing 42 so that the user no longer sees it (“hidden needle injector”). As FIG. 30 shows, in this context latching member 53 provided on adjusting member 105 does not yet reach latch opening 66, and spring 200 is therefore only partially tensioned.

As shown by the enlarged depiction of FIG. 32, in this process clamping jaws 136, 137, which with their teeth 138, 139 (FIG. 5) rest under elastic preload against the tooth set of toothed rod 92, move between protrusions 140, 142 in the interior of housing 42, and clamping jaws 184, 186 of adjusting member 105 slide out of their position between said protrusions 140, 142. What is in operation here is therefore a control system using cams 140, 142 provided in housing 42, which can also be referred to as a gated control system.

In the position shown in FIGS. 31 and 32, both clamping jaws 136, 137 and toothed rod 92 are prevented from moving farther in the distal direction, because clamping jaws 136, 137 are pressed against toothed rod 92 and, as a result of the tooth sets, constitute more or less one unit with it; and because protrusions 140, 142 constitute an axial stop for clamping jaws 136, 137.

On the other hand, upper clamping jaws 184, 186 can now deflect resiliently outward against their spring force acting in the medial direction (i.e. toward toothed rod 92), as shown by FIGS. 33 through 35.

FIGS. 33, 34, and 35 show completion of the motion in the direction of arrow 280, i.e. complete cocking of injection device 40. In this, elastic latch member 53 (on adjusting member 105) snaps into opening 66 of dose-setting part 58. During this operation, clamping jaws 184, 186 of adjusting member 105 slide over an appropriate number of teeth 104 corresponding to the dose that is set. A comparison of FIG. 35 to FIG. 32 shows that in this example, clamping jaws 184, 186 have slid a distance D (four teeth) in the distal direction over toothed rod 92. For better comprehension, the numbers 184′, 186′ in FIG. 35 indicate the position of the clamping jaws according to FIG. 32. During this movement of clamping jaws 184, 186, toothed rod 92 is immobilized by clamping jaws 136, 137, as already described with reference to FIG. 32. The distance D determines the dose that is subsequently injected.

In this operation, a relative displacement thus occurs between actuation knob 44 and the distal end of actuation member 91, which is displaced together with toothed rod 92; this is evident at the top end in FIG. 34.

Injection device 40 is now cocked, the correct dose is set, and the device is ready for injection. As shown in FIG. 36, the patient places the proximal end of injection device 40 onto body part 284 into which injection is to occur, presses in the medial direction (arrow 286 of FIG. 36) on clip 62, and thereby pushes elastic latching member 53 out of latch opening 66. The cocked injection spring 200 then pushes the adjusting member in the proximal direction, and because of the engagement of upper clamping jaws 184, 186 into teeth 104 of toothed rod 92, all of the motion of adjusting member 105 is transferred to toothed rod 92.

Because lower clamping jaws 136, 137 are held, at the start of this proximal motion, in contact against toothed rod 92 by housing cams 140, 142, the beginning of this motion is transferred by direct mechanical coupling to needle 268, so that the latter penetrates into body part 284.

This causes clamping jaws 136, 137 to slide out of housing cams 140, 142 so that they can deflect radially outward against their spring force; as a result, toothed rod 92 then begins to displace piston 90 in cartridge 50 in the proximal direction. The pressure in injection liquid 226′ thus rises, and this increased hydraulic pressure brings about a continuation of the proximal motion of the (already inserted) needle 268. Radial protrusions 80 of cartridge holder 74 thus come into contact against stop 84, thereby stopping the motion of cartridge holder 74. The remaining motion of toothed rod 92, over distance D (FIG. 35), causes the previously set amount of liquid to be expelled out of cartridge 50 into body part 284, where said liquid is labeled 226″ in FIGS. 36 and 37.

If the dose setting remains unchanged, all that is then required is for the user (optionally after exchanging injection needle 268) to bring injection device 40 back into the position shown in FIG. 33 using cocking knob 44, and he or she can then perform another injection. After reconstitution, injection device 40 is therefore extremely easy to use, and presents no difficulties even for patients with poor eyesight.

Numerous variants and modifications are of course possible in the context of the present invention.

1. An injection device,
comprising a housing (42) for receiving a container (50) comprising a liquid (49; 226;);
in which container (50) a piston (90) is displaceably arranged so that by means of pressure upon the distal end of said piston (90), liquid can be expelled from the container (50);
comprising a tappet (92), displaceably arranged in the housing (42), which is configured for contact against the distal end of the piston (90) and which enables transfer of a force to the piston (90) in the proximal direction;
and comprising a member (91), associated with the tappet (92) and displaceable relative thereto, which at its proximal end region can be joined to the piston (90) in such a way that it enables the transfer of a force in the proximal direction and the transfer of a force in the distal direction to the piston (90).

2. The injection device according to claim 1, wherein the displaceable member (91) is equipped at its distal end region with a coupling apparatus (124).

3. The injection device according to claim 2, wherein the coupling apparatus (124) serves for detachable connection to an extension member (126) which is configured in order to extend the displaceable member (91).

4. The injection device according to one or more of the foregoing claims, wherein the displaceable member (91) is arranged displaceably in a recess (114) of the tappet (92).

5. The injection device according to claim 4, wherein the displaceable member (91) is guided nonrotatably in the recess (114) of the tappet (92).

6. The injection device according to claim 4 or 5, wherein at least one stop (FIG. 11: 118, 119) is associated with the displaceable member (91) in order to limit its axial movement relative to the tappet (92).

7. The injection device according to one of the foregoing claims, wherein the tappet is configured as a toothed rod (92).

8. The injection device according to claim 7, wherein an adjusting member (105) is provided which is equipped with at least one resilient ratchet tooth (184′, 186′) for resilient engagement with the teeth (104) of the toothed rod (92).

9. The injection device according to claim 8, wherein the at least one resilient ratchet tooth (184′, 186′) of the adjusting member (105) has associated with it a cam control system (140, 142) which, controlled by the axial position of the adjusting member (105) relative to the housing (42), blocks a resilient deflection of said at least one ratchet tooth (184′, 186′).

10. The injection device according to one of claims 7 through 9, wherein the container (50) is axially displaceable relative to the housing (42) in order to enable, by displacement of the container (50), an insertion motion into the skin of the patient of a hollow needle (268) connected to the container (50).

11. The injection device according to claim 10, wherein at least one resilient ratchet tooth (138, 139) which rests elastically against teeth (104) of the toothed rod (92) is joined to the displaceable container (50).

12. The injection device according to claim 11, wherein the at least one resilient ratchet tooth (138, 139) of the container (50) has associated with it a cam control system (140, 142) which, controlled by the axial position of the container (50) relative to the housing (42), blocks a resilient deflection of said at least one ratchet tooth (138, 139).

13. The injection device according to one of claims 7 through 12, wherein a connection (136, 137) influenced by the axial position of the container (50) relative to the housing (42) is provided between the displaceable container (50) and the toothed rod (92).

14. The injection device according to one of the foregoing claims, wherein a signal member (131) that is actuable by means of the reconstitution operation is provided.

15. The injection device according to one or more of the foregoing claims, having a spring (200) for the storage of energy for an injection, which spring (200) can be cocked by displacement of an adjusting member (105) in the distal direction,
and having a latching member (53, 66) for releasable-latching of the adjusting member (105) in this displaced position.

16. The injection device according to claim 15, wherein at least one cam (140, 142), which in the displaced position of the adjusting member (105) activates a position-dependent mechanical connection (136, 137) between the toothed rod (92) and container (50), is provided in the housing (42).

17. The injection device according to one or more of the foregoing claims, wherein the container (50) has associated with it a holder (74) which is displaceable axially in the housing (42) between a proximal and a distal end position and is configured to receive the container (50).

18. The injection device according to claim 17, wherein the length of the holder (74) is modifiable.

19. The injection device according to claim 18, wherein the holder (74) has a proximal segment (76) and a distal segment (78) which are joined to one another by means of an adjustable connection (77) that enables a modification of the overall length of the holder (74).

20. The injection device according to claim 19, wherein the adjustable connection comprises a micro-ratchet (77) which connects a proximal segment (76) and a distal segment (78) of the holder (74) to one another in adjustable fashion.

21. The injection device according to one or more of claims 17 through 20, wherein the holder (74) is equipped in its distal region with stop means (80) which define its proximal and/or its distal end position relative to the housing (42);
and in which the length adjustment of the holder (74) is accomplished relative to said stop means, so that upon a change in length of the holder (74), the position of the container (50) relative to the toothed rod (92) is modified.

22. The injection device according to one or more of the foregoing claims, wherein a dose-setting part (58) that is displaceable in the longitudinal direction relative to the housing (42) has latch means (66) for snap-locking of a latching element (53).

23. The injection device according to claim 22, wherein a member (62) for releasing the snap-locking between the latch means (66) and the latching element (53) is provided on the displaceable dose-setting part (58).

24. The injection device according to claim 22 or 23, wherein there is provided on the housing (42) a threaded sleeve (52) which is rotatable but not axially displaceable relative to it, which is in engagement with a thread (56) on the displaceable dose-setting part (58).

25. The injection device according to one or more of claims 22 through 24, wherein the displaceable dose-setting part (58) is not rotatable relative to the housing (42).

26. A method for reconstituting an injection liquid in an injection device for subsequent use of the reconstituted injection liquid in said device,
which injection device has
a housing for receiving a cartridge, equipped with a piston, for a liquid;
a tappet for impinging upon said piston in the context of an injection; and
a member that is displaceable relative to said tappet and can be joined to the piston,
having the following steps:
a) a container that contains one component for reconstitution of the injection liquid is brought into liquid communication with the cartridge;
b) by displacement of the displaceable member in a defined direction, liquid is pumped out of the cartridge into said container in order to reconstitute the injection liquid therein;
c) by displacement of the displaceable member in a direction opposite to the defined direction, the reconstituted liquid is sucked back out of the container into the cartridge;
d) the container is removed.

27. The method according to claim 26, wherein in step c) the displaceable member is displaced in the direction opposite to the defined direction until the piston comes into contact against the tappet.

28. The method according to claim 26 or 27, wherein as a result of the action of energy stored in a spring, a hollow injection needle joined to the cartridge is inserted, and injection liquid is injected after the insertion,
insertion of the hollow injection needle by means of the stored energy being accomplished first by mechanical direct drive of the hollow injection needle and then by hydraulic force transfer by means of pressure elevation in the cartridge.
